(19) United States
(12) Patent Application Publication
(10) Pub. No.: US 2016/0136639 A1
(43) Pub. Date: May 19, 2016

(54) Mechanical Separator for a Biological Fluid

(71) Applicant: Becton, Dickinson and Company, Franklin Lakes, NJ (US)

(72) Inventor: Robert J. Losada, Astoria, NY (US)

(21) Appl. No.: 14/629,584

(22) Filed: Feb. 24, 2015

Related U.S. Application Data
(60) Provisional application No. 62/079,230, filed on Nov. 13, 2014.

(51) Int. Cl. B01L 3/00; G01N 33/49

(52) U.S. Cl. CPC B01L 3/50215; G01N 33/491; B01L 2300/0832; B01L 2300/06; B01L 2400/0409

(57) ABSTRACT

A mechanical separator and separation assembly for separating a fluid sample into first and second parts within a collection container is disclosed. The mechanical separator has a body having a through-hole for allowing fluid to pass therethrough and includes a first portion, having a first density, and a second portion, having a second density different from the first density. The body defines a longitudinal axis extending perpendicular to the through-hole, and exhibits a first compression value when a force is applied to the body along this axis. The body also defines an axis extending perpendicular to the longitudinal axis and along the through-hole and exhibits a second compression value when a force is applied along this axis. The first compression value is different than the second compression value and may be less than the second compression value.

### CROSS-REFERENCE TO RELATED APPLICATION

The present application claims priority to U.S. Provisional Application Ser. No. 62/079,230, entitled “Mechanical Separator for a Biological Fluid”, filed Nov. 13, 2014, the entire disclosure of which is hereby incorporated by reference.

### BACKGROUND OF THE INVENTION

1. Field of the Invention

The subject invention relates to a device for separating higher and lower density fractions of a fluid sample. More particularly, this invention relates to a device for collecting and transporting fluid samples whereby the device and fluid sample are subjected to centrifugation in order to cause separation of the higher density fraction from the lower density fraction of the fluid sample.

2. Description of Related Art

Diagnostic tests may require separation of a patient's whole blood sample into components, such as serum or plasma (the lower density phase components), and red blood cells (the higher density phase components). Samples of whole blood are typically collected by venipuncture through a cannula or needle attached to a syringe or an evacuated blood collection tube. After collection, separation of the blood into serum or plasma and red blood cells is accomplished by rotation of the syringe or tube in a centrifuge. In order to maintain the separation, a barrier must be positioned between the higher density and lower density phase components. This allows the separated components to be subsequently examined.

A variety of separation barriers have been used in collection devices to divide the area between the higher density and lower density phases of a fluid sample. The most widely used devices include thixotropic gel materials, such as polyester gels. However, current polyester gel serum separation tubes require special manufacturing equipment to both prepare the gel and fill the tubes. Moreover, the shelf-life of the gel-based separator product is limited. Over time, globules may be released from the gel mass and enter one or both of the separated phase components. Furthermore, commercially available gel barriers may react chemically with the analytes. Accordingly, if certain drugs are present in the blood sample when it is taken, an adverse chemical reaction with the gel interface can occur. Furthermore, if an instrument probe is inserted too deeply into a collection container, then the instrument probe may become clogged if it contacts the gel.

Certain mechanical separators have also been proposed in which a mechanical barrier can be employed between the higher and lower density phases of the fluid sample. Conventional mechanical barriers are positioned between higher and lower density phase components utilizing elevated gravitational forces applied during centrifugation. For proper orientation with respect to plasma and serum specimens, conventional mechanical separators are typically positioned above the collected whole blood specimen prior to centrifugation. This typically requires that the mechanical separator be affixed to the underside of the tube closure in such a manner that blood fill occurs through or around the device when engaged with a blood collection set or phlebotomy needle. This attachment is required to prevent the premature movement of the separator during shipment, handling, and blood draw. Conventional mechanical separators are typically affixed to the tube closure by a mechanical interlock between the bellows component and the closure.

Conventional mechanical separators have some significant drawbacks. As shown in FIG. 1, conventional separators include a bellows 2 for providing a seal with the tube or syringe wall 4. Typically, at least a portion of the bellows 2 is housed within, or in contact with a closure 6. As shown in FIG. 1, as the needle 8 enters through the closure 6, the bellows 2 is depressed. This creates a void 9 in which blood may pool during insertion or removal of the needle. This can result in sample pooling under the closure, device pre-launch in which the mechanical separator prematurely releases during blood collection, trapping of a significant quantity of fluid phases, such as serum and plasma, poor sample quality, and/or barrier failure under certain circumstances. Furthermore, previous mechanical separators are costly and complicated to manufacture due to the complicated multi-part fabrication techniques.

Accordingly, a need exists for a separator device that is compatible with standard sampling equipment and reduces or eliminates the aforementioned problems of conventional separators. A need also exists for a separator device that is easily used to separate a blood sample, minimizes cross-contamination of the higher and lower density phases of the sample during centrifugation, is independent of temperature during storage and shipping, and is stable to radiation sterilization. A need further exists for a unitary separation device that requires fewer relative moving parts and that allows for enhanced ease of introducing a specimen into a collection container.

### SUMMARY OF THE INVENTION

In accordance with an aspect of the present invention, a device for separating a fluid into first and second parts within a container includes a body having a through-hole defined therethrough. The body includes a first portion defining an upper surface of the body, and a second portion defining a lower surface of the body, wherein the first portion and the second portion are interfaced. The body defines a longitudinal axis extending perpendicular to the through-hole, and the body exhibits a first compression value when a force is applied to the body along the longitudinal axis. The body also defines a perpendicular axis extending perpendicular to the longitudinal axis and along the through-hole, and the body exhibits a second compression value when a force is applied to the body along the perpendicular axis. The first compression value is different than the second compression value.

In certain configurations, the first compression value is greater than the second compression value. In certain configurations, the force is exerted to the body during applied rotational force.

In accordance with another aspect of the present invention, a separation assembly for enabling separation of a fluid into first and second phases includes a collection container having a first end, a second end, and a sidewall extending therebetween defining an interior. The separation assembly also includes a separator body having a through-hole defined therethrough. The body includes a first portion defining an upper surface of the body, and a second portion defining a lower surface of the body, wherein the first portion and the second portion are interfaced. The separator body defines a longitudinal axis extending between the upper surface and the lower surface, wherein the separator body exhibits a first compression value when a force is applied to the separator body along the longitudinal axis. The separator body also defines a perpendicular axis extending perpendicular to the longitudinal axis, wherein the separator body exhibits a second compression value when a force is applied to the body along the perpendicular axis. The first compression value is different than the second compression value.

In certain configurations, the first compression value is greater than the second compression value. In certain configurations, the force is exerted to the body during rotational force applied to the container.

### BRIEF DESCRIPTION OF THE DRAWINGS

FIG. 1 is a partial cross-sectional side view of a conventional mechanical separator.

FIG. 2 is a perspective view of a mechanical separator in accordance with an embodiment of the present invention.

FIG. 3 is a front view of the mechanical separator of FIG. 2.

FIG. 4 is a cross-sectional view of the mechanical separator of FIG. 2 taken along the longitudinal axis L of the mechanical separator as shown in FIG. 3.

FIG. 5 is a top view of the mechanical separator of FIG. 2.

FIG. 6 is a side view of the mechanical separator of FIG. 2.

FIG. 7 is a cross-sectional view of the mechanical separator of FIG. 2 taken along the longitudinal axis L of the mechanical separator as shown in FIG. 6.

FIG. 8 is a partial cross-sectional side view of a mechanical separator disposed within a collection container in an initial position for allowing fluid to pass through a through-hole in accordance with an embodiment of the present invention.

FIG. 9a is a partial cross-sectional side view of a mechanical separator disposed within a collection container in an intermediate position during centrifugation for allowing fluid to pass around the mechanical separator in accordance with an embodiment of the present invention.

FIG. 9b is a partial cross-sectional front view of the mechanical separator disposed within the collection container of FIG. 9a in the intermediate position during centrifugation for allowing fluid to pass around the mechanical separator in accordance with an embodiment of the present invention.

FIG. 10a is a partial cross-sectional side view of the mechanical separator disposed within a collection container of FIG. 9a in a sealed position after centrifugation in accordance with an embodiment of the present invention.

FIG. 10b is a partial cross-sectional front view of the mechanical separator disposed within a collection container of FIG. 9a in a sealed position after centrifugation in accordance with an embodiment of the present invention.

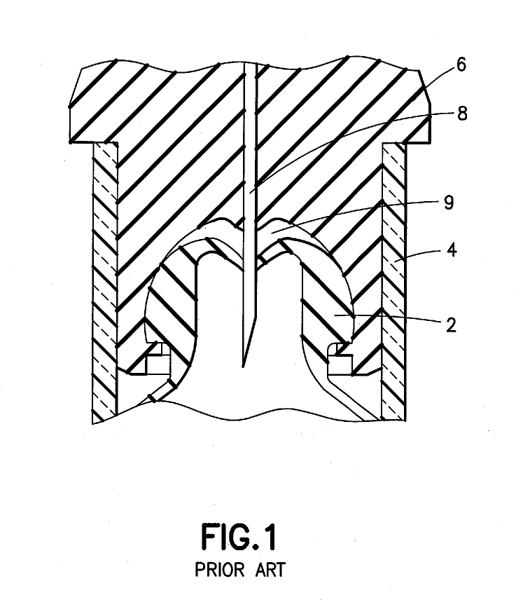

### DETAILED DESCRIPTION OF THE EMBODIMENTS

For purposes of the description hereinafter, the words “upper”, “lower”, “right”, “left”, “vertical”, “horizontal”, “top”, “bottom”, “lateral”, “longitudinal”, and like spatial terms, if used, shall relate to the described embodiments as oriented in the drawing figures. However, it is to be understood that many alternative variations and embodiments may be assumed except where expressly specified to the contrary. It is also to be understood that the specific devices and embodiments illustrated in the accompanying drawings and described herein are simply exemplary embodiments of the invention.

The mechanical separator of the present invention is intended for use with a collection container for providing separation of a sample into higher and lower density phase components, as will be discussed herein. For example, the present mechanical separator can be used to provide a separation of serum or plasma from whole blood through the use of differential buoyancy to cause a sealing area to contract when submerged in a specimen exposed to elevated gravitational forces through applied rotational force or centrifugation. In one embodiment, the elevated gravitational forces can be provided at a rate of at least 2,000 revolutions/minute, such as 3,400 revolutions/minute.

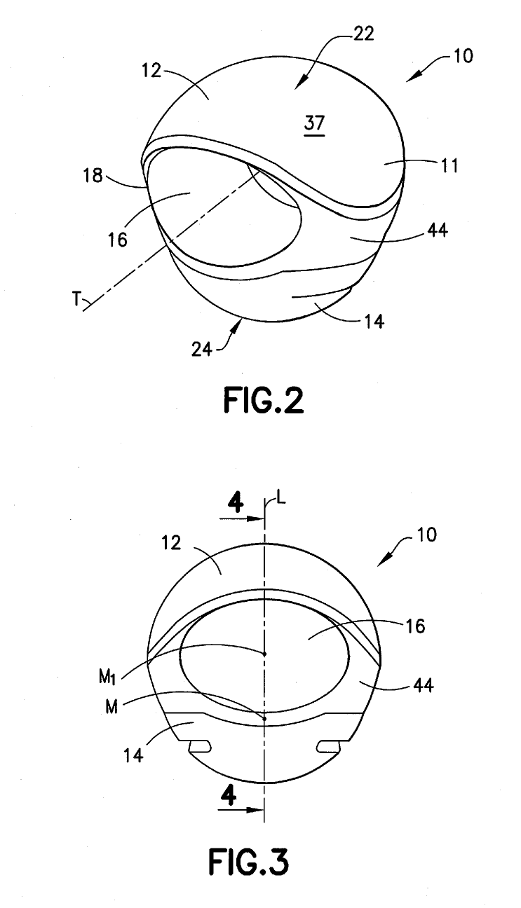

Referring to FIGS. 2-7, a mechanical separator 10 of the present invention includes a separator body 11 including a first portion 12 and a second portion 14 connected to the first portion 12. The first portion 12 has a first density and the second portion 14 has a second density, with the second density being different from the first density and, preferably, greater than the first density. Alternatively or in addition, the first portion 12 has a first buoyancy and the second portion 14 has a second buoyancy, with the second buoyancy being different from the first buoyancy, and, preferably, less than the first buoyancy.

One of the first portion 12 or the second portion 14 of the mechanical separator 10 may be extruded and/or molded of a resiliently deformable and self-sealable material, such as a thermoplastic elastomer (TPE). Alternatively, one of the first portion 12 or the second portion 14 of the mechanical separator 10 may be extruded and/or molded of a resiliently deformable material that exhibits good sealing characteristics when contact is established with a collection container, as will be discussed herein. Maintenance of the density within the specified tolerances is more easily obtained by using a standard material that does not require compounding with, for example, hollow glass micro-spheres in order to reduce the material density. The other of the first portion 12 or the second portion 14 of the mechanical separator 10 can be formed from mineral filled polypropylene.

One of the first portion 12 or the second portion 14 of the mechanical separator 10 is made from a material having a density that is less than the less-dense phase intended to be separated into two phases. For example, if it is desired to separate serum or plasma from human blood, then it is desirable that one of the first portion 12 or the second portion 14 have a density of no more than about 1.020 g/cc.

The other of the first portion 12 or the second portion 14 of the mechanical separator 10 is made from a material having a higher density than the more-dense phase intended to be separated into two phases. For example, if it is desired to separate serum or plasma from human blood, then it is desirable that the other of the first portion 12 or the second portion 14 have a density of at least 1.105 g/cc. It is anticipated herein that both the first portion 12 and the second portion 14 may be formed of various other materials with sufficient biocompatibility, density stability, additive compatibility, and neutrality to analyte interactions, adsorption, and leachability.

The mechanical separator 10 also includes a through-hole 16 defined therein, such as along a through-axis T of the separator body 11. As shown in FIGS. 2, 4, and 6, the through-hole 16 may extend through the entire separator body 11 and includes a first opening 18 and a second opening 20 aligned along the through-axis T. The through-hole 16 may bisect or substantially bisect the volumetric center of the separator body 11. The through-hole 16 may be defined by the first portion 12 or at least a portion of the first portion 12 and at least a portion of the second portion 14.

The first portion 12 has an exterior surface 22 that is generally arcuate in shape, such as at least partially rounded or substantially rounded. The second portion 14 also includes an exterior surface 24 that is also generally arcuate in shape, such as at least partially rounded or substantially rounded. When taken together, the exterior surface 22 of the first portion 12 and the exterior surface 24 of the second portion 14 form a generally round exterior. It is understood herein that the term “round exterior” includes configurations, in addition to a perfect sphere, that are aspects of the invention which may provide slightly non-uniform diameters taken through the mid-point. For example, different planes taken through the first portion 12 and second portion 14 which bisect the midpoint of the mechanical separator 10 may have varying diameters and still give rise to a generally rounded or ball-like mechanical separator 10.

Due to the differential densities of the first portion 12 and the second portion 14, the mechanical separator 10 includes a center of mass M that is offset from the center of volume M1 of the separator body 11, as shown in FIG. 3. Specifically, the volume of the separator body 11 accounted for by the first portion 12 may be significantly greater than the volume of the separator body 11 accounted for by the second portion 14. Accordingly, the center of mass M and center of volume M1 of the separator body 11 may be offset from the center of the through-hole 16.

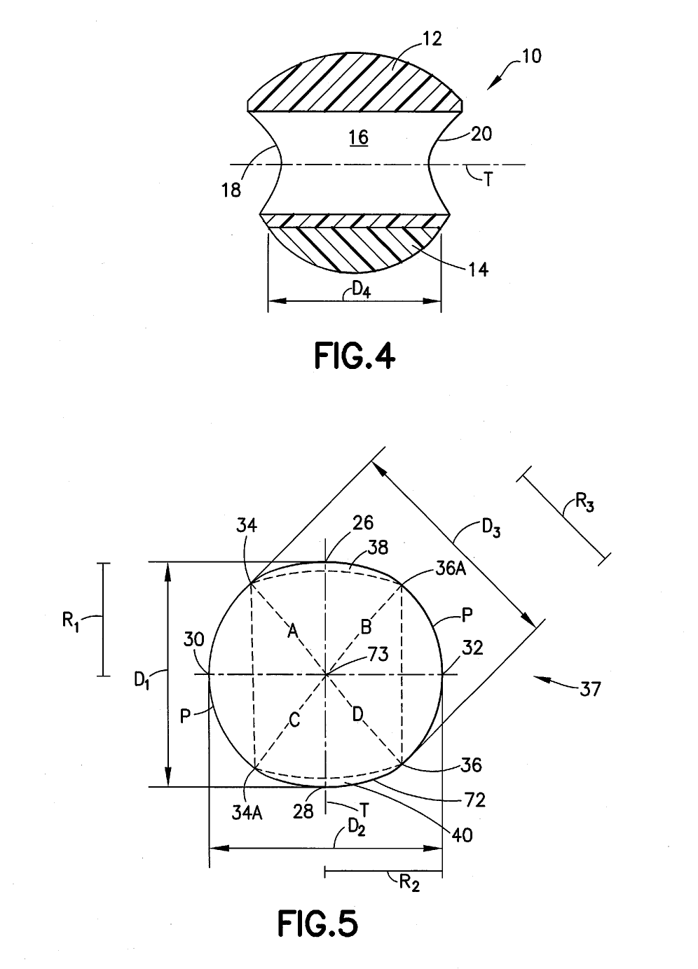

As shown in FIG. 5, the top profile of the separator body 11 may be non-circular. The diameter D₁ of the separator body 11, specifically the first portion 12, taken across the first portion 12 in the direction along the through-axis T of the through-hole 16 and extending between vertically outermost opposing tangent points 26, 28 of the perimeter P of the separator body 11 is less than the diameter D₂ of the separator body 11, specifically the first portion 12, taken across the first portion 12 in the direction perpendicular to the through-axis T of the through-hole 16 and extending between laterally outermost opposing tangent points 30, 32 of the perimeter P of the separator body 11. In addition, the diameter D₃ of the separator body 11, specifically the first portion 12, taken across the first portion 12 at an angle substantially 45° to the through-axis T of the through-hole 16 and extending between diagonally outermost endpoints 34, 36 of the perimeter P of the separator body 11, may be larger than the diameter of the through-hole 16, and is greater than the diameters D₁ and D₂ of the separator body 11. The diameter D₄ of the second portion 14 taken across the second portion 14 along the through-axis T of the through-hole 16, as shown in FIG. 4, may be less than any of the diameters D₁, D₂, or D₃ of the separator body 11.

Referring to FIG. 5, a two-dimensional projection of the top profile of the first portion 12 of the separator body 11 onto a plane may be symmetrical about an orientation plane extending between vertically outermost opposing tangent points 26, 28 of the perimeter of the separator body 11 and from a top surface 37 of the first portion 12 to a bottom surface of the second portion 14 and in the direction of the through-axis T of the through-hole 16. The two-dimensional projection of the top profile of the first portion 12 of the separator body 11 onto a plane may also be symmetrical about an orientation plane extending between laterally outermost opposing tangent points 30, 32 of the perimeter P of the separator body 11 and from the top surface 37 of the first portion 12 to the bottom surface of the second portion 14 and perpendicular to the direction of through-axis T of the through-hole 16. A two-dimensional projection of the top profile of the first portion 12 of the separator body 11 onto a plane may be asymmetrical about an orientation plane extending between diagonally outermost endpoints 34, 36 of the perimeter P of the separator body 11 and from the top surface 37 of the first portion 12 to the bottom surface of the second portion 14 and in a direction diagonal to at least a part of the through-axis T of the through-hole 16. Accordingly, a two-dimensional projection of the top profile of the body 11 onto a plane may be asymmetric about an orientation plane extending between diagonally outermost endpoint 34A, 36A of the perimeter P of the separator body 11 and from the top surface 37 of the first portion 12 to the bottom surface of the second portion 14 and in a direction diagonal to at least a part of the through-axis T of the through-hole 16.

Further, the top profile of the separator body 11 defines a perimeter P that bounds four quadrants A, B, C, D, respectively defined by the intersection of a vertical axis extending between vertically outermost opposing tangent points 26, 28 of the perimeter P of the separator body 11 and a lateral axis extending between laterally outermost opposing tangent points 30, 32 of the perimeter P of the separator body 11. Each quadrant A, B, C, D is substantially bisected by an orientation axis extending between diagonally outermost endpoints 34, 36 or 34A, 36A of the perimeter P of the separator body 11 and bounded by the perimeter P of the separator body 11 as shown in FIG. 5. A two-dimensional projection of the top profile of the separator body 11 onto a plane may be symmetrical about D₁ and D₂ but may be asymmetrical with respect to D₃.

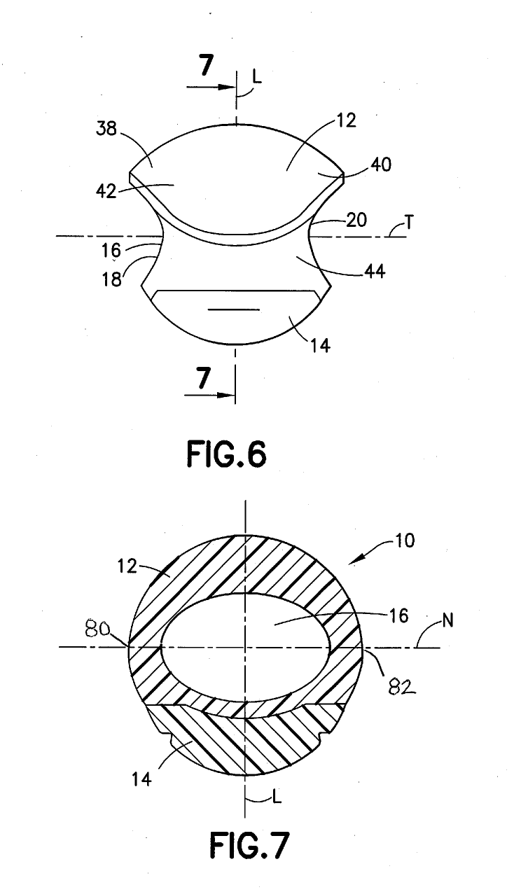

Thus, a top surface 37 of the first portion 12 includes a first extended portion 38 adjacent the first opening 18 of the through-hole 16 defined by tangent point 26, endpoint 34, and endpoint 36A and a second extended portion 40 adjacent the second opening 20 of the through-hole 16 defined by tangent point 28, endpoint 36, and endpoint 34A, that taken with an upper portion 42 (FIG. 6) of the first portion 12, form a substantially non-circular convex top surface 37 of the first portion 12.

As a result, the resistance to compression (compression value) or extension (extension value) of the separator body 11 to forces exerted along a longitudinal axis L of the separator body 11 (shown in FIGS. 3 and 6) extending from the top surface 37 of the first portion 12 to the bottom surface of the second portion 14 and perpendicular to the through-axis T of the through-hole 16 or exerted along the lateral axis N extending from a side 80 to a side 82 and perpendicular to the through-axis T of through-hole 16 is different from, and preferably less than, the resistance to compression (compression value) or extension (extension value) of the separator body 11 to forces along the through-hole axis T of the separator body 11 (shown in FIG. 6) extending from the first opening 18 of the through-hole 16 to the second opening 20 of the through-hole 16. The difference in compression values may be particularly noticeable when the force is a squeezing force.

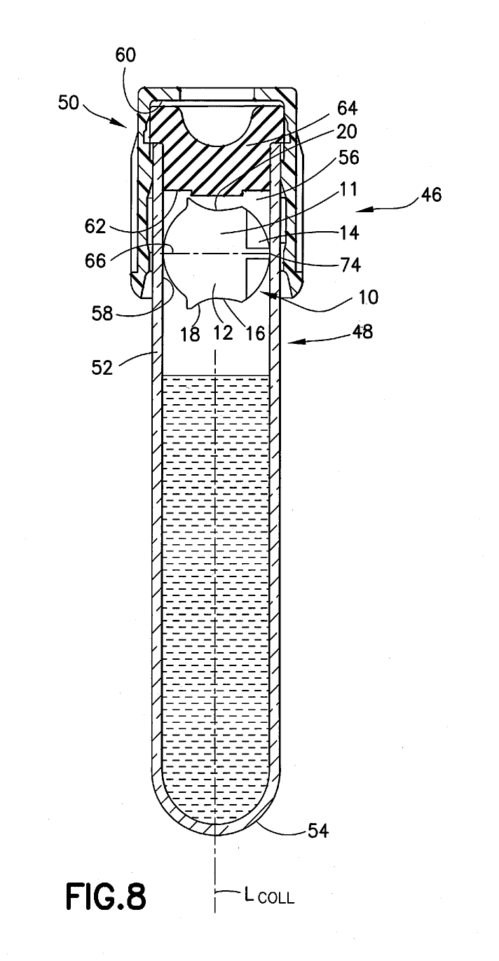

As shown in FIGS. 8-10b, the mechanical separator 10 of the present invention may be provided as a portion of a separation assembly 46 for separating a fluid sample into first and second phases within a collection container 48 having a closure 50. Specifically, the collection container 48 may be a sample collection tube, such as a proteomics, molecular diagnostics, chemistry sample tube, blood, or other bodily fluid collection tube, coagulation sample tube, hematology sample tube, and the like. The collection container 48 includes a closed bottom end 54, an open top end 56, and a cylindrical sidewall 52 extending therebetween. The cylindrical sidewall 52 includes an inner surface 58 with an inside diameter extending substantially uniformly from the open top end 56 to a location substantially adjacent the closed bottom end 54 along the longitudinal axis L_{COLL} of the collection container 48.

Desirably, collection container 48 is an evacuated blood collection tube. The collection container 48 may contain additional additives as required for particular testing procedures, such as protease inhibitors, clotting agents, and the like. Such additives may be in particle or liquid form and may be sprayed onto the cylindrical sidewall 52 of the collection container 48 or located at the bottom 54 of the collection container 48.

The collection container 48 may be made of one or more than one of the following representative materials: polypropylene, polyethylene terephthalate (PET), glass, or combinations thereof. The collection container 48 can include a single wall or multiple wall configurations. Additionally, the collection container 48 may be constructed in any practical size for obtaining an appropriate biological sample. For example, the collection container 48 may be of a size similar to conventional large volume tubes, small volume tubes, or microliter volume tubes, as is known in the art. In one particular embodiment, the collection container 48 may be a standard 13 ml evacuated blood collection tube, as is also known in the art.

The open top end 56 is structured to at least partially receive the closure 50 therein to form a liquid impermeable seal. The closure 50 includes a top end 60 and a bottom end 62 structured to be at least partially received within the collection container 48. Portions of the closure 50 adjacent the top end 56 of the collection container 48 define a maximum outer diameter which exceeds the inside diameter of the collection container 48. The closure 50 includes a pierceable resealable septum 64 penetrable by a needle cannula (not shown). Portions of the closure 50 extending upwardly from the bottom end 62 may taper from a minor diameter which is approximately equal to, or slightly less than, the inside diameter of the collection container 48 to a major diameter that is greater than the inside diameter of the collection container 48 at the top end 60 of the closure 50. Thus, the bottom end 62 of the closure 50 may be urged into a portion of the collection container 48 adjacent the open top end 56 of the collection container 48. The inherent resiliency of closure 50 can insure a sealing engagement with the inner surface 58 of the cylindrical sidewall 52 of the collection container 48. In one embodiment, the closure 50 can be formed of a unitarily molded elastomeric material, having any suitable size and dimensions to provide sealing engagement with the collection container 48. Optionally, the closure 50 may be at least partially surrounded by a shield, such as a Hemogard® Shield commercially available from Becton, Dickinson and Company.

As shown in FIG. 8, the mechanical separator 10 of the present invention may be oriented within the collection container 48 in an initial position in which the through-hole 16 of the mechanical separator 10 is aligned with the open top end 56 of the collection container 48. In the initial position, the through-hole 16 is adapted for allowing fluid to pass therethrough, such as from a needle cannula (not shown) which has pierced the pierceable septum 64 of the closure 50 and is provided in fluid communication with the interior of the collection container 48. The mechanical separator 10 may also be releasably engaged with a portion of the closure 50.

Referring to FIG. 8, the initial open position of the through-hole 16 is substantially aligned with the longitudinal axis L_{COLL} of the collection container 48. The mechanical separator 10 forms an interference engagement with the sidewall 52 of the collection container 48 along a first perimeter 66 as shown in FIG. 8. During specimen draw into the collection container 48, the initial separator position minimizes the accumulation of blood between the mechanical separator 10 and the closure 50. This reduces the formation of clots and/or fibrin strands which may disrupt function of the mechanical separator 10.

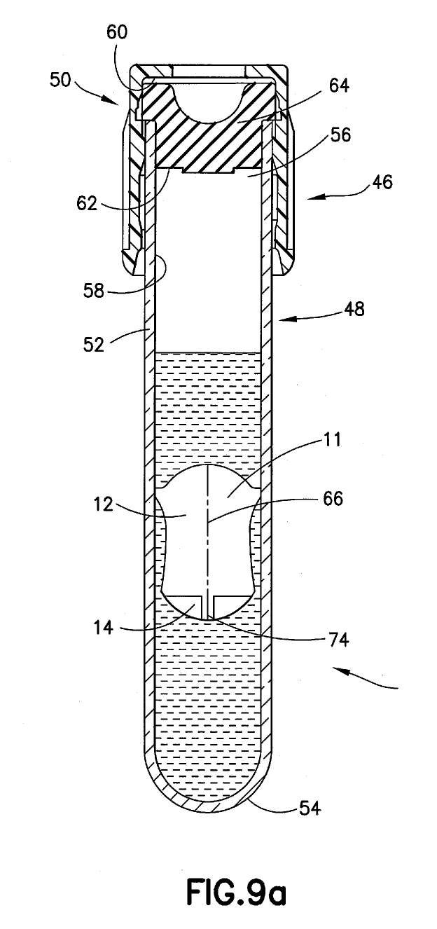

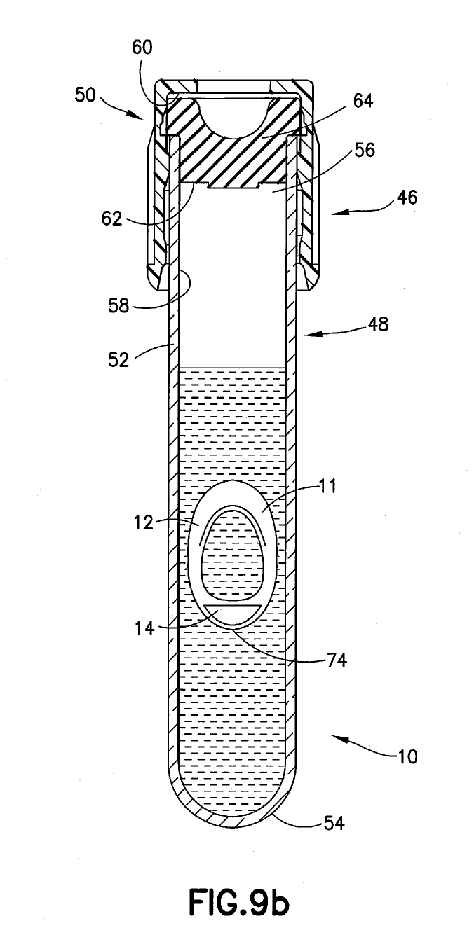

Upon application of rotational force, such as during centrifugation, and transition of the mechanical separator 10 as shown in FIGS. 9a and 9b, the mechanical separator 10 experiences a rotational moment, deforms sufficiently to disengage from engagement with the collection container 48, and rotates approximately 90°. In the case shown in FIGS. 8-10b, where the density of the first portion 12, such as a float, is less than the density of the second portion 14, such as a ballast, the mechanical separator 10 will be oriented with the second portion 14 facing the bottom end 54 of the collection container 48.

Once the mechanical separator 10 contacts the fluid contained within the collection container 48, air that occupies the through-hole 16 is progressively displaced by the fluid as the device submerges. When the mechanical separator 10 is submerged in the fluid, the difference in the buoyancy between the first portion 12 and the second portion 14 generates a differential force across the mechanical separator 10. During centrifugation, the differential force causes the separator body 11 to elongate along the longitudinal axis L_{COLL} of the collection container and contract away from the sidewall 52 of the collection container 48 along the lateral axis N, thereby reducing the effective diameter of the separator body 11 and opening a communicative pathway for the flow of fluid, such as higher and lower density phase components, past the separator body 11. It is noted that the first portion 12 may be adapted for deformation in a direction substantially perpendicular to the through-hole 16.

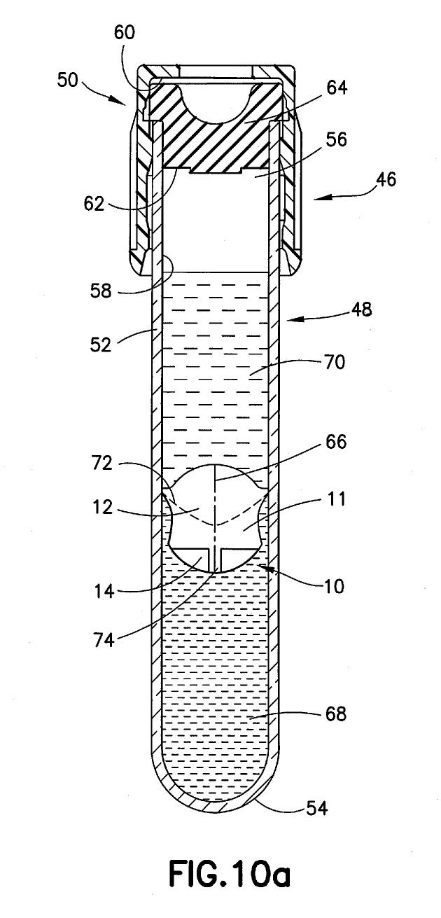

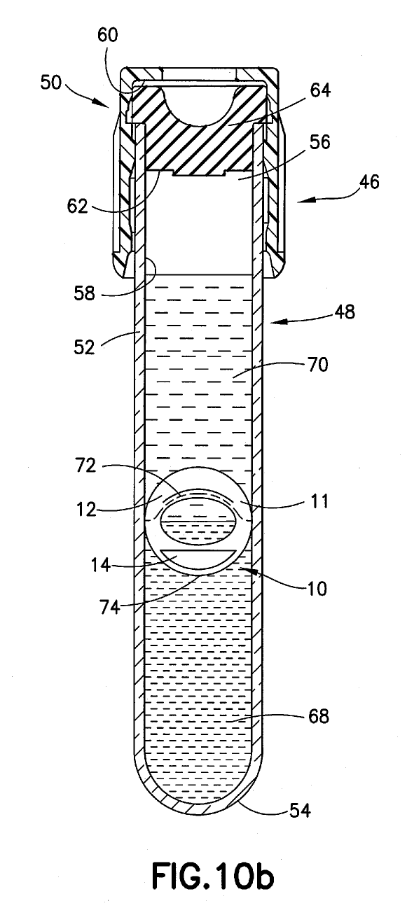

Once separation of the lower and higher density phases is complete and the application of rotational force has ceased, the mechanical separator 10 becomes oriented in a sealing position (as shown in FIGS. 10a and 10b) between a separated higher density phase 68 and a separated lower density phase 70. At the same time, the elongation of the separator body 11 ceases, causing the separator body 11 to return to its initial configuration, thereby forming a seal between a second outer perimeter 72 of the first portion 12 and the inner surface 58 of the sidewall 52 of the collection container 48. The outer perimeter 72 has an outer circumference that is at least slightly larger than the interior circumference of the sidewall 52 of the collection container 48. In addition, the smallest diameter D₁ of the top surface 37 of the first portion 12 is at least slightly greater than the diameter of the inner surface 58 of the collection container 48. Accordingly, the mechanical separator 10 is adapted to prevent fluid from passing between or around the separator body 11 and the collection container 48, and also prevents fluid from passing through the through-hole 16, effectively establishing a barrier and the second sealing perimeter 72 between higher and lower density phases within the sample.

The difference in compression and expansion values of the mechanical separator in the direction of the through-hole (separator T axis) versus the direction perpendicular to the through-hole (such as along the separator L and N axes) allows the separator to elongate in the longitudinal direction and contract in the lateral direction during the application of rotational force while maintaining a stabilizing separator contact with the tube inner surface 58 of sidewall 52 along the separator through-hole direction. This stabilizing contact assists in the proper movement and orientation of the separator during centrifugation. It also ensures that, upon cessation of rotational forces, the separator moves up, rather than down, to form a sealing engagement, or barrier, with the tube inner surface 58 of sidewall 52 thereby reducing the potential for contamination of the separated low density phase by the high density phase.

As can be determined from the discussions above, the separator body 11 is in a compressed, but substantially unstressed state when it forms a seal with the inner surface 58 of the sidewall 52 of the collection container 48. The shape of the top profile of the separator body 11 provides for this compression to form a tight seal with the inner surface 58 of the sidewall 52 of the collection container 48. The inner surface 58 of the sidewall 52 of the collection container 48 forms a first perimeter 66 shape and engagement with the separator that is substantially circular, while the separator body 11 has a top surface that defines a second perimeter 72 shape that is non-circular and provides a non-circular engagement with the inner surface 58 of sidewall 52 of collection container 48 as shown in FIGS. 10a and 10b.

In order to form a tight seal between the separator body 11 and inner surface 58 of the sidewall 52 of the collection container 48, in the substantially unstressed condition, the second perimeter 72 of the separator body 11 defines a radial distance R₁ from a center 73 of the top surface of separator body 11 that is greater than the corresponding radius of the inner surface 58 of the sidewall 52 of the collection container 48 (FIG. 5). The first radial distance R₁ may correspond to half the diameter D₁ shown in FIG. 5 and may be defined along a plane extending along the through-axis T of the through-hole 16 and perpendicular to the longitudinal axis of separator body 11 and passing through the center of the through-hole 16. In addition, the second perimeter 72 of the separator body 11 defines at least a second radial distance R₂ from the center 73 of the top surface of separator body 11 that is greater than the corresponding radius of the inner surface 58 of the sidewall 52 of the collection container 48. The second radial distance R₂ may be half the diameter D₂ shown in FIG. 5 and may be defined along a plane extending perpendicular to the through-hole 16 and longitudinal axis of separator body 11 and passing through the center of the through-hole 16. The second radial distance R₂ may be greater than the first radial distance R₁. Further, the second perimeter 72 of the separator body 11 may define a third radial distance R₃ from the center 73 of the top surface of separator body 11 that is greater than the corresponding radius of the inner surface 58 of the sidewall 52 of the collection container 48. This radial distance R₃ may be half the diameter D₃ shown in FIG. 5 and may be defined along a plane representing a substantially 45° angle between the first radial distance R₁ and the second radial distance R₂ and perpendicular to the longitudinal axis of separator body 11. The third radial distance R₃ may be greater than the first radial distance R₁ and the second radial distance R₂.

In the stressed condition, the second perimeter 72 of the separator body 11 defines another radial distance R₂ from the center 73 of the top surface of separator body 11 that is slightly less than or equal to the corresponding radius of the inner surface 58 of the sidewall 52 of the collection container 48 as the separator body 11 is elongated along the longitudinal axis L and contracted along the lateral axis N during the application of rotational forces. Also, it should be noted that in the stressed and deformed condition, the second perimeter 72 of separator body 11 continues to define a radial distance R₁ from center 73 of the top surface of separator body 11 that, unlike R₂, continues to be greater than the corresponding radius of the inner surface 58 of sidewall 52 of the collection container 48.

Referring to FIGS. 8-10b, the mechanical separator 10 may include an initial engagement band 74 circumferentially disposed about the separator body 11. The initial engagement band 74 may be disposed about the separator body 11 in a direction substantially perpendicular to the through-hole 16. The initial engagement band 74 may be continuously provided about the separator body 11, or may optionally be provided in segments about the separator body 11. The first portion 12 and the initial engagement band 74 may be formed from the same material, such as TPE. The initial engagement band 74 may be provided such that a first portion of the first portion 12 forms the initial engagement band 74, and a second portion substantially bisects the second portion 14.

As shown specifically in FIG. 8, the initial engagement band 74 provides an interference engagement between the separator body 11 and the inner surface 58 of the collection container 48. In this configuration, a first perimeter 66 about the separator body 11 is inline with the initial engagement band 74. This first perimeter 66 assists in maintaining the separator body 11 in proper alignment with the open top end 56 of the collection container 48, such that fluid entering the collection container 48 from a cannula (not shown) disposed through the pierceable septum 64 will pass through the first opening 18 of the through-hole 16, through the through-hole 16, and out the second opening 20 of the through-hole 16.

The invention claimed is:

1. A device for separating a fluid into first and second parts within a container, comprising:
a body having a through-hole defined therethrough, the body comprising:
a first portion defining an upper surface of the body; and
a second portion defining a lower surface of the body, wherein the first portion and the second portion are interfaced,
wherein the body defines a longitudinal axis extending perpendicular to the through-hole, and wherein the body exhibits a first compression value when a force is applied to the body along the longitudinal axis,
wherein the body defines a perpendicular axis extending perpendicular to the longitudinal axis and along the through-hole, and wherein the body exhibits a second compression value when a force is applied to the body along the perpendicular axis, and
wherein the first compression value is different than the second compression value.

2. The device of claim 1, wherein the first compression value is greater than the second compression value.

3. The device of claim 1, wherein the force is exerted to the body during applied rotational force.

4. A separation assembly for enabling separation of a fluid into first and second phases, comprising:
a collection container having a first end, a second end, and a sidewall extending therebetween defining an interior; and
a separator body having a through-hole defined therethrough, the body comprising:
a first portion defining an upper surface of the body; and
a second portion defining a lower surface of the body, wherein the first portion and the second portion are interfaced,
wherein the separator body defines a longitudinal axis extending between the upper surface and the lower surface, and wherein the separator body exhibits a first compression value when a force is applied to the separator body along the longitudinal axis,
wherein the separator body defines a perpendicular axis extending perpendicular to the longitudinal axis and along the through-hole, and wherein the separator body exhibits a second compression value when a force is applied to the body along the perpendicular axis, and
wherein the first compression value is different than the second compression value.

5. The separation assembly of claim 4, wherein the first compression value is greater than the second compression value.

6. The separation assembly of claim 4, wherein the force is exerted to the body during rotational force applied to the container.

7. The separation assembly of claim 6, wherein the applied rotational force causes the separator body to elongate in a longitudinal direction and contract in a lateral direction.

8. The separation assembly of claim 7, wherein the separator body adjacent the through-hole maintains contact with the interior of the sidewall during applied rotational force.
